(19) United States
(12) Patent Application Publication
(10) Pub. No.: US 2026/0263744 A1
(43) Pub. Date: Sep. 10, 2026

(54) IMPLANT DELIVERY ASSEMBLIES

(71) Applicant: Covidien LP, Mansfield, MA (US)

(72) Inventor: Ramon CARRILLO, Santa Ana, CA (US)

(21) Appl. No.: 19/552,186

(22) Filed: Feb. 27, 2026

Related U.S. Application Data
(60) Provisional application No. 63/769,417, filed on Mar. 10, 2025.

(51) Int. Cl. A61M 25/00; A61B 17/12

(52) U.S. Cl. CPC A61M 25/0082; A61M 25/0068; A61M 25/008; A61B 2017/12054; A61M 2205/0266

(57) ABSTRACT

An example implantation assembly is configured to deploy an implantable device. The implantation assembly may include a pusher and a wire. The pusher defines a pusher lumen extending between a proximal pusher end and a distal pusher end. The distal pusher end defines a pusher opening. The wire extends within the pusher lumen. A distal portion of the wire defines a pair of hooked prongs. The pusher opening is configured to receive a retention portion of the implantable device into the pusher lumen. The pair of hooked prongs is configured to releasably secure the retention portion of the implantable device within the pusher lumen.

### CROSS-REFERENCE TO RELATED APPLICATIONS

This application claims the benefit of U.S. Provisional Patent Application Serial No. 63/769,417, filed Mar. 10, 2025, the entire content of which is incorporated herein by reference.

### TECHNICAL FIELD

The present disclosure generally relates to assemblies and techniques for delivering implantable devices to an implantation site.

### BACKGROUND

Implantable devices may be transported to and deployed at a target site (e.g., through a catheter to a cavity or vessel of a patient). For example, an implantable device may be coupled to a wire, and the wire may be advanced through a catheter to transport the implantable device.

### SUMMARY

In general, this disclosure describes assemblies and techniques for delivering implantable devices to a target site. For example, an implantation assembly may be used to advance an implantable device to the target site, and the implantable device may be released from the implantation assembly at the target site. For example, the implantation assembly may releasably secure the implantable device to a wire in a pusher via a mechanical coupling. When the implantable assembly is in position such that the implantable device is at the target site, a force may be applied to the wire to cause the wire to retract from the implantable device to release the implantable device. The mechanical detachment of the implant may be relatively simpler to use compared to other implant detachment systems, for example, electrical detachment of an implant from an implantable assembly.

In some examples, an example implantation assembly is configured to deploy an implantable device. The implantation assembly may include a pusher and a wire. The pusher defines a pusher lumen extending between a proximal pusher end and a distal pusher end. The distal pusher end defines a pusher opening. The wire extends within the pusher lumen. A distal portion of the wire defines a pair of hooked prongs. The pusher opening is configured to receive a retention portion of the implantable device into the pusher lumen. The pair of hooked prongs is configured to releasably secure the retention portion of the implantable device within the pusher lumen.

In some examples, an example technique includes advancing an implantation assembly to an implantation site. The implantable assembly may include a pusher and a wire. The pusher defines a pusher lumen extending between a proximal pusher end and a distal pusher end. The distal pusher end defines a pusher opening. The wire extends within the pusher lumen. A distal portion of the wire defines a pair of hooked prongs. The pusher opening is configured to receive a retention portion of the implantable device into the pusher lumen. The pair of hooked prongs is configured to releasably secure a retention portion of the implantable device within the pusher lumen. The technique may further include retracting the wire proximally relative to the implantable device to release the retention portion from the pair of hooked prongs by causing the pair of hooked prongs to slide proximally about the retention portion.

In some examples, an example technique includes receiving a distal portion of a wire within a pusher lumen defined by a pusher. The pusher lumen extends between a proximal pusher end and a distal pusher end. The distal pusher end defines a distal pusher opening. The distal portion of the wire defines a pair of hooked prongs. The technique may further include receiving a retention portion of an implantable device through the distal pusher opening into the pusher lumen. The technique may further include releasably securing the retention portion of the implantable device between the pair of hooked prongs.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1A is a conceptual diagram illustrating a partial side view of an example implantation assembly including a pusher and a wire releasably secured to an implantable device.

FIG. 1B is a conceptual diagram illustrating a partial side view of the implantation assembly of FIG. 1A (without the pusher) with the wire releasably secured to the implantable device.

FIG. 1C is a conceptual diagram illustrating a top view of a distal pusher end of the pusher of the assembly of FIG. 1A defining a pusher opening.

FIG. 1D is a conceptual diagram illustrating a partial front view of the implantation assembly of FIG. 1A with the wire defining a pair of hooked prongs.

FIG. 1E is a conceptual diagram illustrating a partial rear view of the implantation assembly of FIG. 1A.

FIG. 1F is a conceptual diagram illustrating a partial top view of the assembly of FIG. 1A with the retention portion of the implantable device releasably secured to the pair of hooked prongs of the wire.

FIG. 1G is a conceptual diagram illustrating a partial top view of the assembly of FIG. 1A in a proximal direction at plane P-P.

FIG. 2 is a cross-sectional view of an example wire including a flat portion.

FIG. 3 is a cross-sectional view of an example wire including a curved portion.

FIG. 4 is a cross-sectional view of an example wire including a round portion.

FIG. 5A is a conceptual diagram illustrating a partial rear view of the implantation assembly of FIG. 1A (without the pusher) in an initial configuration showing the pair of hooked prongs ready to be released from the retention portion of the implantable device.

FIG. 5B is a conceptual diagram illustrating a partial rear view of the implantation assembly of FIG. 5A in an intermediate configuration with the pair of hooked prongs sliding proximally about the retention portion of the implantable device.

FIG. 5C is a conceptual diagram illustrating a partial rear view of the implantation assembly of FIG. 5A in a final configuration with the retention portion of the implantable device released and spaced from the pair of hooked prongs of the wire.

FIG. 6 is a conceptual diagram illustrating a partial rear view of an example implantation assembly including an angled pair of hooked prongs.

FIG. 7 is a conceptual diagram illustrating a partial side view of an example implantation assembly including a linear pair of hooked prongs.

FIG. 8 is a conceptual diagram illustrating a partial side view of an example implantation assembly including a V-shaped pair of hooked prongs.

FIG. 9 is a conceptual diagram illustrating a partial top view of an example implantation assembly including a curved pair of hooked prongs.

FIG. 10A is a conceptual diagram illustrating a partial side view of an initial manufacturing configuration of the implantation assembly of FIG. 1A.

FIG. 10B is a conceptual diagram illustrating a partial side view of an intermediate manufacturing configuration of the implantation assembly of FIG. 11A.

FIG. 10C is a conceptual diagram illustrating a partial side view of a final manufacturing configuration of the implantation assembly of FIG. 11A.

FIG. 11 is a conceptual diagram illustrating a partial side view of an example pusher including a pusher cap secured to a pusher body.

FIG. 12A is a conceptual diagram illustrating a partial side view of an initial manufacturing configuration of an example implantation assembly including the pusher of FIG. 11.

FIG. 12B is a conceptual diagram illustrating a partial side view of a first intermediate manufacturing configuration of the implantation assembly of FIG. 12A.

FIG. 12C is a conceptual diagram illustrating a partial side view of a second intermediate manufacturing configuration of the implantation assembly of FIG. 12A.

FIG. 12D is a conceptual diagram illustrating a partial side view of a third intermediate manufacturing configuration of the implantation assembly of FIG. 12A.

FIG. 13 is a flow diagram illustrating an example technique for delivering an implantable device to an implantation site using an implantation assembly.

FIG. 14 is a flow diagram illustrating an example technique for forming an implantation assembly.

### DETAILED DESCRIPTION

The present disclosure describes assemblies and techniques for delivering implantable devices to an implantation site. In some examples, assemblies and techniques according to the present disclosure may be used to advance and mechanically release an implantable device at the implantation site. For example, an implantation assembly may include a wire including a pair of hooked prongs releasable secured to a retention portion of the implantable device. In some examples, the wire is a flat nitinol wire. Each hooked prong of the pair of hooked prongs may be U-shaped, and the pair of hooked prongs is spaced by an inter-prong slot. The wire may be attached to a rip-cord. Exerting a jerk or a pulling force on the rip-cord may cause the pair of hooked prongs to slide about the retention portion and release the implantable device from the wire.

In some examples, an example implantation assembly includes a pusher tube defining a pusher lumen, and a wire extending through the pusher lumen. A distal portion of the wire may define a pair of hooked prongs (e.g., U-shaped hooked prongs). The pair of hooked prongs define a slot therebetween. The pair of hooked prongs may extend along an axial direction of the distal portion. A U-shape (or other hooked forms or shapes) may facilitate engaging a retention portion of an implantable device between the pair of hooked prongs.

The retention portion of the implantable device may include a ball configured to be secured between the pair of hooked prongs. To detach the implant from the wire, the wire is pulled in a proximal direction away from the implant. For example, a proximal jerk may be applied, causing the pair of hooked prongs to slide proximally about the ball and allow the ball to be released. After the implantable device is released, the wire may be retracted proximally, leaving the implantable device in place. Thus, a mechanical coupling may be used to secure the implantable device to the wire for positioning and advancing the implantable device to an implantation site, and a mechanical force may be used to release the implantable device from the wire at the implantation site.

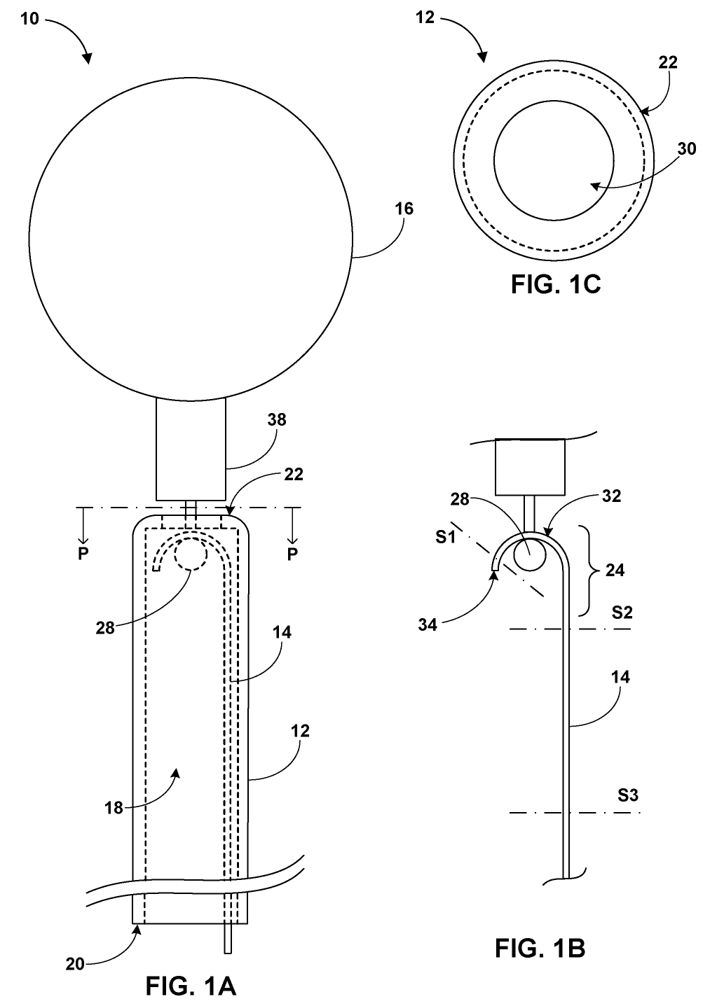

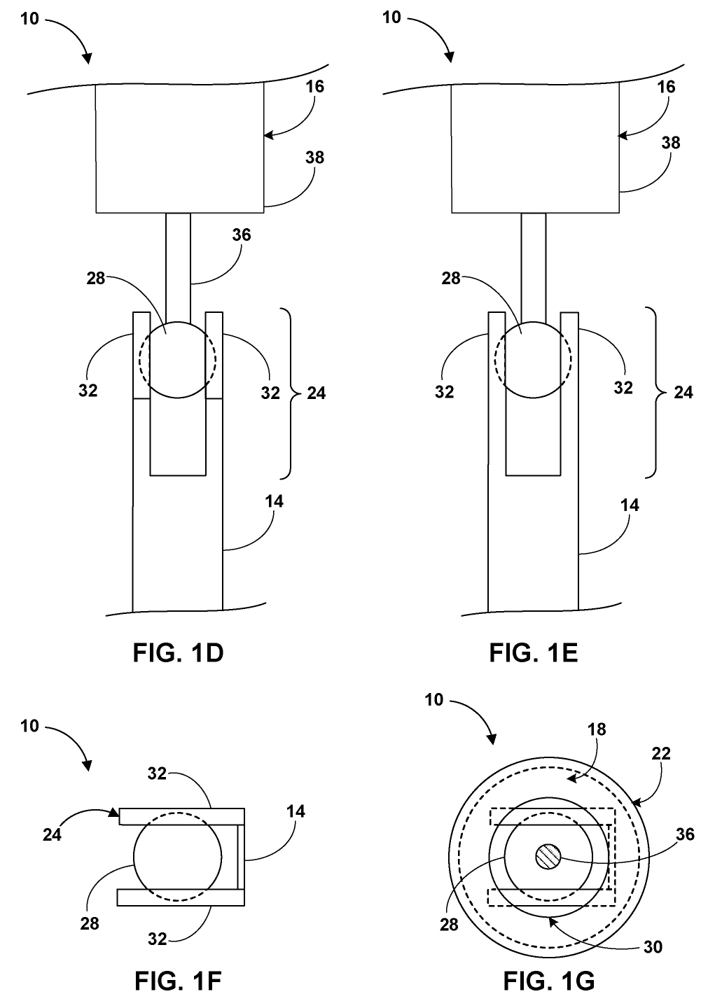

FIG. 1A is a conceptual diagram illustrating a partial side view of example implantation assembly 10 including a pusher 12 and a wire 14 releasably secured to an implantable device 16. Implantable device 16 may include any suitable implant. For example, implantable device 16 may include an implantable body comprising one or both of a mesh (e.g., a spherical mesh) or a coil (e.g., a helical coil). Implantation assembly 10 is configured to deploy implantable device 16 at a target site. For example, implantable device 16 may be releasably secured to wire 14, and pusher 12 may be advanced to position implantable device 16 at the target site.

Pusher 12 defines a pusher lumen 18 extending between a proximal pusher end 20 and a distal pusher end 22. Pusher 12 may include any suitable composition, for example, a biocompatible polymer. A portion of or an entirety of pusher 12 may be opaque, translucent, or transparent. Pusher 12 is configured to retain wire 14 within pusher lumen 18 during advancing implantable device 16 to the target site. For example, at least a portion of wire 14 extends within pusher lumen 18.

Wire 14 may include any suitable material that is sufficiently rigid to be pushed or pulled, e.g., along with or relative to pusher 12 or implantable device 16. In some examples, wire 14 includes a metal or an alloy. The alloy may include a shape-memory alloy (e.g., nitinol). In some examples, wire 14 consists of or consists essentially of (e.g., excepting for minor impurities) nitinol.

FIG. 1B is a conceptual diagram illustrating a partial side view of implantation assembly 10 of FIG. 1A (without the pusher) with wire 14 releasably secured to implantable device 16. A distal portion 24 of wire 14 defines a pair of hooked prongs 32. For example, each hooked prong of pair of hooked prongs 32 may be unitary with wire 14. In some examples, pair of hooked prongs 32 may be formed by machining, stamping, cutting, or laser ablating a slot in wire 14. In some examples, distal portion 24 of wire 14 is bent to a predetermined hooked shape after or before the slot is formed in wire 14 to define pair of hooked prongs 32. In other examples, one or both of pair of hooked prongs 32 are separate formed and secured to wire 14 (e.g., by a weld). Thus, a composition of pair of hooked prongs 32 may be identical to or different from that of wire 14. Pair of hooked prongs 32 of wire 14 is releasably secured to implantable device 16, for example, to a retention portion 28 of implantable device 16.

Retention portion 28 may have any suitable shape or size. In some examples, retention portion 28 includes a ball configured to be releasably secured by pair of hooked prongs 32. However, retention portion 28 may have any suitable shape, having any polygonal or curved surface slidable along pair of hooked prongs 32. Retention portion 28 may have any suitable composition, for example, including any suitable rigid material. In some examples, retention portion 28 includes a metal or an alloy, for example, a steel. In some examples, wire 14 and/or pair of hooked prongs 32 includes nitinol and retention portion 28 includes the steel.

In some examples, at least a portion of pair of hooked prongs 32 is U-shaped, as seen in FIGS. 1A and 1B. The U-shape may facilitate some relative movement between wire 14 and implantable device 16 while implantable device 16 is advanced along potentially curved, tortuous, or winding paths or bends to the target site. At the same time, the U-shape may facilitate a mechanical securement of retention portion 28 between pair of hooked prongs 32, for example, preventing a movement of retention portion 28 distally beyond pair of hooked prongs 32.

FIG. 1C is a conceptual diagram illustrating a top view of pusher 12 of assembly 10 of FIG. 1A with distal pusher end 22 of pusher 12 defining a pusher opening 30. Pusher opening 30 is configured to receive retention portion 28 of implantable device 16 into pusher lumen 18.

FIG. 1D is a conceptual diagram illustrating a partial front view of implantation assembly 10 of FIG. 1A with wire 14 including pair of hooked prongs 32. FIG. 1E is a conceptual diagram illustrating a partial rear view of implantation assembly 10 of FIG. 1A. Pair of hooked prongs 32 is configured to releasably secure retention portion 28 of implantable device 16 within pusher lumen 18. For example, at least a portion of retention portion 28 engages at least a portion of pair of hooked prongs 32, such that contact between at least the portion of retention portion 28 that engages with at least the portion of pair of hooked prongs 32 causes retention portion 28 to be restrained between pair of hooked prongs 32.

Each prong of pair of hooked prongs 32 may be identical in size and shape. In other examples, one prong of pair of hooked prongs 32 may differ from the other prong of pair of hooked prongs 32 in size, shape, or contour.

FIG. 1F is a conceptual diagram illustrating a partial top view of assembly 10 of FIG. 1A with retention portion 28 of implantable device 16 releasably secured to pair of hooked prongs 32 of wire 14. As seen in FIG. 1F, in addition to front and rear portions of pair of hooked prongs 32 shown in FIGS. 1D and 1E, top portions of pair of hooked prongs 32 may also restrain retention portion 28 between or within pair of hooked prongs 32. In particular, the top portions of pair of hooked prongs 32 may restrain retention 28 against a distal movement past pair of hooked prongs 32.

FIG. 1G is a conceptual diagram illustrating a partial top view of assembly 10 of FIG. 1A in a proximal direction at plane P-P. As seen in FIG. 1G, pusher opening 30 may be wider than a maximum dimension of retention portion 28. For example, pusher opening 30 may be configured to receive retention portion 28 of implantable device 16 within pusher lumen 18. However, retention portion 28 may yet be restrained within pusher lumen 18 by pair of hooked prongs 32. For example, pusher opening 30 may be narrower than pair of hooked prongs 32 (for example, a widest edge-to-edge distance between outermost edges of pair of hooked prongs 32) to resist movement of pair of hooked prongs 32 (and thus retention portion 28) distally beyond pusher opening 30.

While pair of hooked prongs 32 is configured to secure implantable device 16 to wire 14 while implantable device 16 is advanced to the target site, pair of hooked prongs 32 is configured to allow implantable device 16 to be released from wire 14 at the target site. For example, the composition of wire 14 or pair of hooked prongs 32 (which may be the same or different from each other) may be rigid, yet flexible. The rigidness may allow wire 14 to push implantable device 16 to desired location once pair of hooked prongs 32 engages retention port 28. The flexibility allows retraction of pair of hooked prongs about retention portion in response to a force that exceeds a predetermined threshold. Therefore, a proximal force exceeding the predetermined threshold (e.g., a jerk) applied to wire 14 causes pair of hooked prongs 32 to proximally retract about (e.g., by sliding) retention portion 28, releasing retention portion 28 from pair of hooked prongs 32. Thus, assembly 10 may be used to advance and position implantable device 16 while being secured to wire 14, and then release implantable device 16 from wire 14 at the target site.

In the example shown in FIGS. 1A to 1G, pair of hooked prongs 32 defines a pair of prong ends 34 spaced apart from each other. In other examples, pair of hooked prongs 32 defines a pair of prong ends secured to each other (e.g., by a bridge), while leaving an inter-prong spacing between remaining portions of the pair of hooked prongs.

Implantable device 16 may include a retention wire 36 coupled to retention portion 28. For example, pair of hooked prongs 32 is configured to slidably receive retention wire 36 of retention portion 28 of implantable device 16. Thus, at least a portion of retention wire 36 may be narrower than an inter-prong spacing between pair of hooked prongs 32. Retention wire 36 may have any suitable width or length, and may be configured to permit a predetermined range of motion between retention portion 28 and pair of hooked prongs 32. While retention wire 36 has a circular cross-section in the example shown in FIG. 1G, retention wire 36 may have any suitable cross-section in other examples.

Each hooked prong of pair of hooked prongs may independently have any suitable length relative to a length of retention wire 36. In some examples, at least one hooked prong of pair of hooked prongs has a prong length that is at least 10%, or at least 20%, or at least 50%, or at least 100%, or at least 150%, or at least 200%, greater than the length of retention wire 36. In some examples, the prong length is at least twice the length of retention wire 36. Each hooked prong of pair of hooked prongs may independently have any suitable length relative to a maximum dimension of retention portion 28. In some examples, the prong length is at least 10%, or at least 20%, or at least 50%, or at least 100%, or at least 150%, or at least 200%, greater than the maximum dimension of retention portion 28. In some examples, the prong length is at least twice the maximum dimension of retention portion 28 (e.g., of a width of a ball in examples in which retention portion 28 includes the ball).

Pair of hooked prongs 32 may have any suitable total prong width (e.g., an edge-to-edge width between outermost edges of pair of hooked prongs 32) relative to a maximum dimension of retention portion 28. In some examples, the total prong width is at least 10%, or at least 20%, or at least 50%, or at least 100%, or at least 150%, or at least 200%, greater than the maximum dimension of retention portion 28. In some examples, the total prong width is at least twice the maximum dimension of retention portion 28 (e.g., of a width of a ball in examples in which retention portion 28 includes the ball).

Pair of hooked prongs 32 may have any suitable total prong width (e.g., an edge-to-edge width between outermost edges of pair of hooked prongs 32) relative to a maximum wire width of wire 14. In some examples, the total prong width is at least 50%, at least 80%, at least 100%, at least 120%, or at least 150%, or at least 200%, of maximum wire width of wire 14. In some examples, the total prong width is the same as the maximum wire width of wire 14.

Each prong of pair of hooked prongs 32 may independently have any suitable prong thickness relative to a maximum dimension of retention portion 28. In some examples, the prong thickness is at least 10%, at least 20%, or at least 50%, of the maximum dimension of retention portion 28. In some examples, the prong thickness is no more than 10%, or no more than 20%, or no more than 50%, of the maximum dimension of retention portion 28.

Each prong of pair of hooked prongs 32 may independently have any suitable prong thickness relative to a maximum wire thickness of a remaining portion of wire 14. In some examples, the prong thickness is at least 10%, at least 20%, at least 50%, at least 100%, at least 120%, or at least 150%, of the wire thickness of the remaining portion of wire 14. In some examples, the prong thickness is no more than 150%, no more than 120%, no more than 100%, no more than 50%, or no more than 20%, of the wire thickness of the remaining portion of wire 14.

Wire 14 may have any suitable wire thickness relative to a maximum wire width. In some examples, the wire thickness is at least 5%, 10%, or at least 20%, of the maximum wire width of wire 14. In some examples, the wire thickness is no more than 20%, no more than 10%, or no more than 5%, of the maximum wire width of wire 14.

Implantable device 16 may further include an engagement portion 38 configured to engage against distal pusher end 22 of pusher 12. For example, engagement portion 38 may be distal to engagement portion 28 or retention wire 36, such that engagement portion 38 may engage externally against distal pusher end 22 when engagement portion 28 is received within pusher lumen 18 of pusher 12. In some examples, pusher opening 30 is narrower than at least engagement portion 38 of implantable device 16 to resist entry of engagement portion 38 into pusher lumen 18 through pusher opening 30. In some examples, engagement portion 38 includes a radiopaque marker band.

Wire 14 may have any suitable cross-section along various portions of wire 14. For example, wire 14 may have a first cross-section S1 along pair of hooked prongs 32, a second cross-section S2 adjacent a transition of pair of hooked prongs 32 into a body of wire 14, and a third cross-section S3 in the body of wire 14. The first, second, and third cross-sections S1, S2, S3 may differ in one or both of size or shape, or may be identical in size or shape. One or both of a width (e.g., a lateral width) or a thickness (or any dimension) of wire 14 may be constant or vary along a portion or an entirety of wire 14.

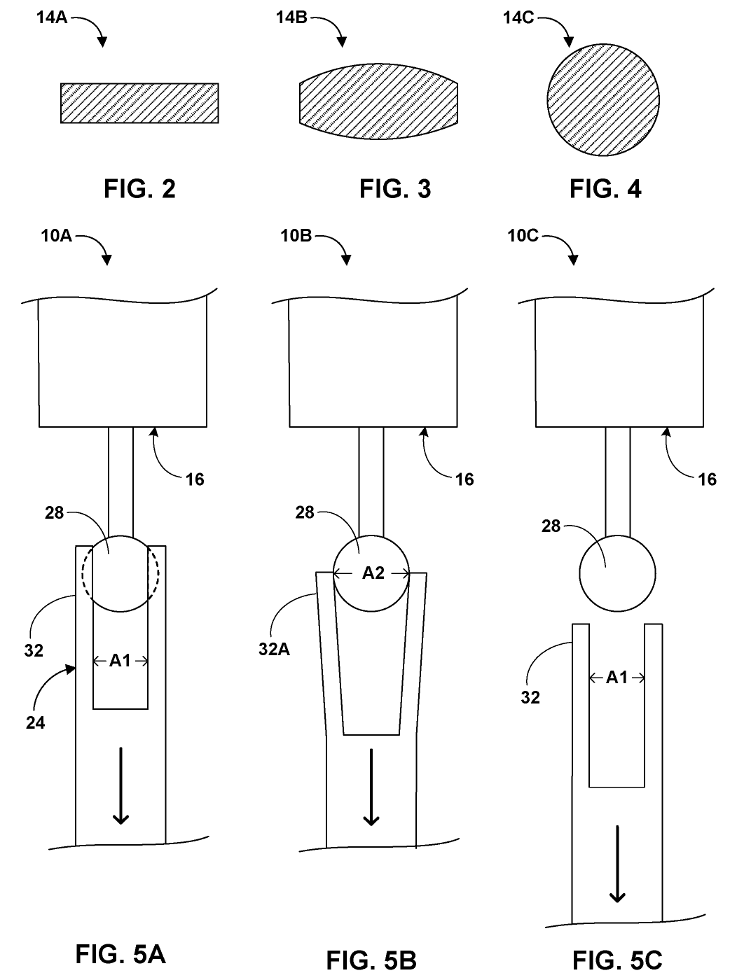

FIG. 2 is a cross-sectional view of an example wire 14A including a flat portion. FIG. 3 is a cross-sectional view of an example wire 14B including a curved portion (e.g., a bulging or lenticular cross-section). FIG. 4 is a cross-sectional view of an example wire 14C including a round portion (e.g., a circular or ellipsoidal cross-section). At least a portion (less than an entirety or an entirety) of wire 14 may define a flat wire cross-section (e.g., similar to FIG. 2) or a curved or round wire cross-section (e.g., similar to FIGS. 3 or 4). In some examples, at least one prong of pair of hooked prongs 32 defines a flat cross-section (e.g., similar to FIG. 2 at S1). In some such examples, a remaining portion of wire 14 defines a curved or round wire cross-section (e.g., similar to FIGS. 3 or 4).

Different configurations of assembly 10 in course of release of implantable device from wire 14 is described with reference to FIGS. 5A to 5C. In particular, pair of hooked prongs 32 is laterally movable between a first configuration 32 in which retention portion 28 of implantable device 16 is secured between pair of hooked prongs 32 and a second configuration 32A in which retention portion 28 of implantable device 16 is releasable from pair of hooked prongs 32.

FIG. 5A is a conceptual diagram illustrating a partial rear view of implantation assembly 10 of FIG. 1A (without the pusher) in an initial configuration 10A showing pair of hooked prongs 32 ready to be released from retention portion 28 of implantable device 16. A force is applied to wire 14 in a proximal direction, causing pair of hooked prongs 32 to engage against retention portion 28, and initially tending to pull retention portion 28 in a proximal direction. Engagement portion 38 of implantable device 16 may engage against an external surface of distal pusher end 22, holding implantation device 16 in engagement against pusher 12 while wire 14 is proximally retracted within pusher lumen 18 of pusher 12. As the force is continued to be applied, pair of hooked prongs 32 may begin to deform about retention portion 28.

FIG. 5B is a conceptual diagram illustrating a partial rear view of the implantation assembly of FIG. 5A in an intermediate configuration 10B of pair of hooked prongs 32A sliding proximally about retention portion 28 of implantable device 16. Intermediate configuration of pair of hooked prongs 32A is laterally wider relative to initial configuration of pair of hooked prongs 32, with an increase in the inter-prong spacing from an initial maximum spacing of A1 to an intermediate maximum spacing of A2. For example, initial maximum spacing A1 of initial configuration of pair of hooked prongs 32 is less than a maximum dimension of retention portion 28 (restraining retention portion 28), and intermediate maximum spacing A2 of intermediate configuration of pair of hooked prongs 32A is equal to the maximum dimension of retention portion 28 (releasing retention portion 28).

Pair of hooked prongs 32 defines first maximum inter-prong spacing A1 in a first (initial) configuration. For example, pair of hooked prongs 32, in a first configuration in which retention portion 28 of the implantable device is secured between pair of hooked prongs 32A, defines a first maximum inter-prong spacing A1 in a range of 30% to 70% of a maximum dimension of retention portion 28 (e.g. a width of a ball in which retention portion 28 includes the ball). In some examples, the first maximum inter-prong spacing A1 is in a range of 30% to 70% of a maximum lateral width of pair of hooked prongs 32 in the first configuration.

First maximum inter-prong spacing A1 may be determined relative to a maximum lateral width of wire 14 or of total prong width of pair of hooked prongs 32 (e.g., end-to-end lateral width between outermost edges). In some examples, inter-prong spacing A1 is in a range from 20% to 80%, or 30% to 70%, or 40% to 60%, or a maximum lateral width of wire 14 or of pair of hooked prongs 32. In some examples, inter-prong spacing A1 is at least 10%, at least 20%, at least 30%, at least 40%, at least 50%, at least 60%, at least 70%, or at least 80%, or at least 90%, of a maximum lateral width of wire 14 or of pair of hooked prongs 32. In some examples, inter-prong spacing A1 is no more than 10%, no more than 20%, no more than 30%, no more than 40%, no more than 50%, no more than 60%, no more than 70%, no more than 80%, or no more than 90%, of a maximum lateral width of wire 14 or of pair of hooked prongs 32.

Pair of hooked prongs 32A defines a second maximum inter-prong spacing A2 greater than the first maximum inter-prong spacing A1 in a second (intermediate) configuration. Pair of hooked prongs 32, in the second configuration in which retention portion 28 of implantable device 16 is releasable from pair of hooked prongs 32A, defines second maximum inter-prong spacing A2 that is at least equal to the maximum width of retention portion 28 (e.g. a width of a ball in which retention portion 28 includes the ball). Second maximum inter-prong spacing A2 is greater than first maximum inter-prong spacing A1. For example, second maximum inter-prong spacing A2 may be at least 1%, at least 5%, at least 10%, at least 15%, or at least 20% greater than first maximum inter-prong spacing A1.

As the force is continued to be applied to wire 14, pair of hooked prongs 32 continues to slide against retention portion 28 and ultimately converges back to the initial configuration with inter-prong spacing A1.

FIG. 5C is a conceptual diagram illustrating a partial rear view of the implantation assembly 10 of FIG. 5A in a final configuration 10C with retention portion 28 of the implantable device released and spaced from pair of hooked prongs 32 of wire 14. Thus, in final configuration 10C, pair of hooked prongs 32 converge back to their initial configurations from intermediate configuration 32A shown in FIG. 5B. One or both of pusher 12 or wire 14 can now be proximally retracted away from the target site, leaving implantable device 16 in place at the target site.

Thus, assembly 10 may be used to advance and release implantable device 16 at any predetermined target site accessible by advancing pusher 12.

In the examples described with reference to assembly 10, pair of hooked prongs 32 are substantially parallel to wire 14 in an initial configuration in which implantable device 16 is secured to wire 14. For example, wire 14 extends along a wire path, and at least one prong of pair of hooked prongs 32 extends along a path parallel to at least a portion of the wire path. In other examples, at least one prong of a pair of hooked prongs extends along a path diverging laterally away from at least a portion of a wire path.

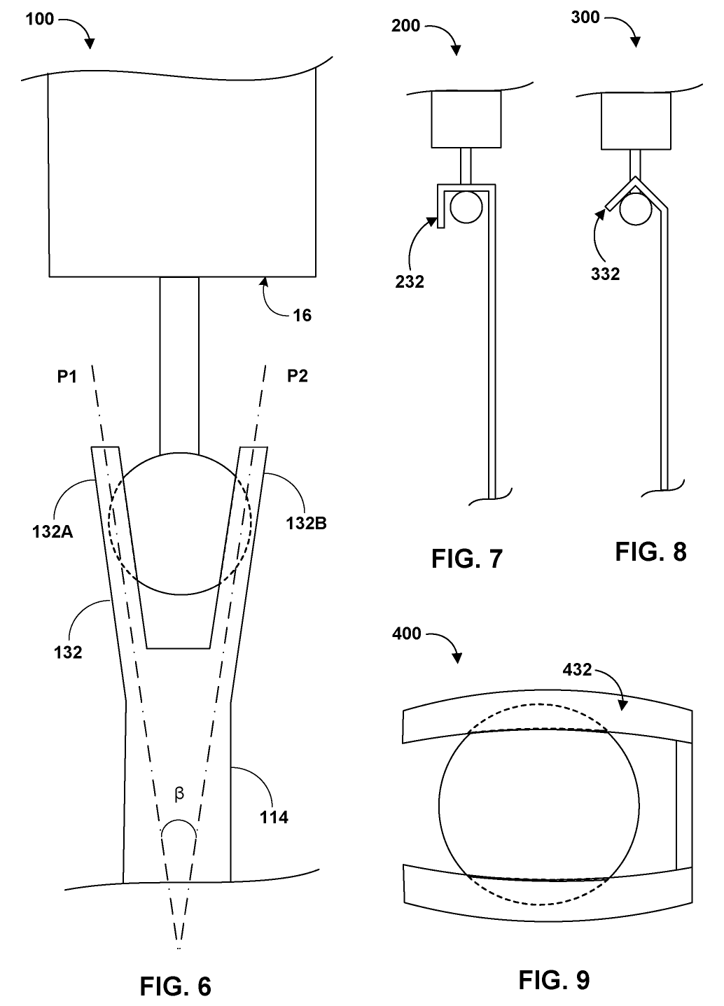

FIG. 6 is a conceptual diagram illustrating a partial rear view of an example implantation assembly 100 including an angled pair of hooked prongs 132. Assembly 100 and components thereof are similar to assembly 10 and like components described with reference to FIGS. 1A to 1G, except for the differences described with reference to FIG. 6. For example, a first prong 132Aof pair of hooked prongs 132 extends along a first plane P1, and a second prong 132 of the pair of hooked prongs extends along a second plane P2. In some examples, second plane P2 is parallel to first plane P1. In other examples, second plane P2 is angled relative to first plane P1. For example, first and second planes P1 and P2 may subtend a predetermined angle β. Thus, first prong 132A and second prong 132B may diverge laterally away from a wire 114 along angled planes P1 and P2.

Portions of pair of hooked prongs 32 or 132 may extend along curved or linear paths, as described with reference to FIGS. 7 to 9.

FIG. 7 is a conceptual diagram illustrating a partial side view of an example implantation assembly 200 including a linear pair of hooked prongs 232. Assembly 200 and components thereof are similar to assembly 10 and like components described with reference to FIGS. 1A to 1G, except for the differences described with reference to FIG. 7. In linear pair of hooked prongs 232, at least one prong of pair of hooked prongs 232 extends along at least a partially linear path. For example, the partially linear path may include linear segments connected by any suitable angles. In some such examples, an entirety of the at least one prong extends along a linear path. In some such examples, each prong of pair of hooked prongs 232 extends along at least the partially linear path. For example, each prong of pair of hooked prongs 232 extends along a linear path segment that is perpendicular to neighboring linear path segments. Thus, in the example shown in FIG. 7, each prong of pair of hooked prongs 232 extends along three linear path segments.

FIG. 8 is a conceptual diagram illustrating a partial side view of an example implantation assembly 300 including V-shaped pair of hooked prongs 332. Assembly 300 and components thereof are similar to assembly 200 and like components described with reference to FIG. 7, except for the differences described with reference to FIG. 8. Each prong of pair of hooked prongs 332 extends along three linear path segments. In V-shaped pair of hooked prongs 332, two upper linear path segments are perpendicular to each other, while a third vertical path segment is angled at 120º relative to the upper linear path segments.

FIG. 9 is a conceptual diagram illustrating a partial top view of an example implantation assembly 400 including a curved pair of hooked prongs 432. Assembly 400 and components thereof are similar to assembly 10 and like components described with reference to FIGS. 1A to 1G, except for the differences described with reference to FIG. 9. At least one prong of pair of hooked prongs 432 extends along at least a partially curved path. For example, at least one prong of pair of hooked prongs 432 is laterally curved. In some such examples, an entirety of the at least one prong extends along a curved path. In some such examples, each prong of pair of hooked prongs 432 extends along a curved path.

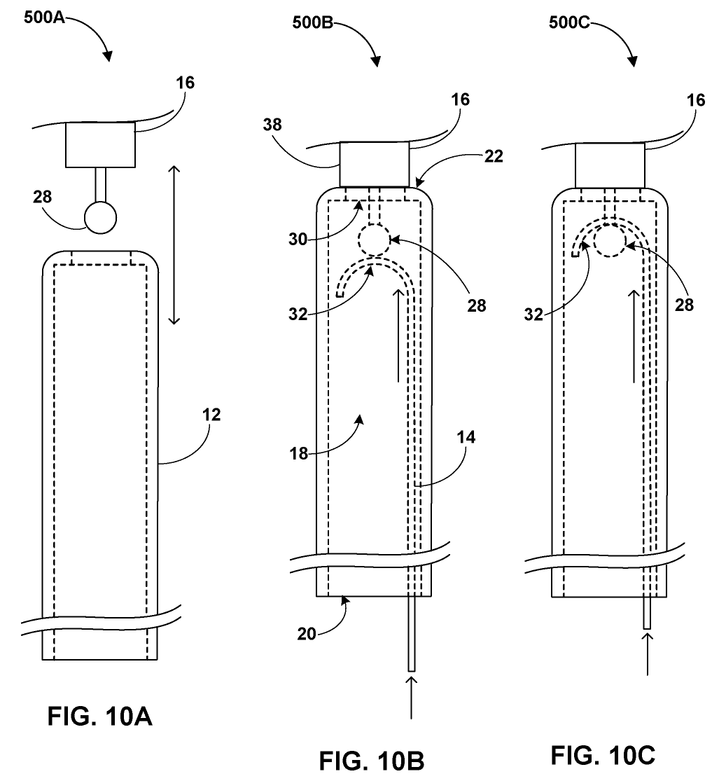

Various assemblies according to the present disclosure may be formed using any suitable techniques. For example, prior to use by a clinician, a retention portion of an implantable device may be releasably secured to a wire within a pusher lumen of a pusher using any suitable technique. Some examples of techniques for forming an implantation assembly are described with reference to FIGS. 10A to 12B.

FIG. 10A is a conceptual diagram illustrating a partial side view of an initial manufacturing configuration 500A of implantation assembly 10 of FIG. 1A. In initial manufacturing configuration 500A, retention portion 28 is ready to be releasably secured to wire 14 within pusher 12. Wire 14 and retention portion 28 may be introduced within pusher lumen 18 of pusher 12 in any suitable order. For example, wire 14 may be introduced into pusher lumen 18 in a distal direction from an opening in proximal pusher end 20, or in a proximal direction from pusher opening 30 in distal pusher end 22. Before or after introducing wire 14, retention portion 28 may be introduced within pusher lumen 18 in a proximal direction from pusher opening 30 in distal pusher end 22.

FIG. 10B is a conceptual diagram illustrating a partial side view of an intermediate manufacturing configuration 500B of the implantation assembly 10 of FIG. 10A. In intermediate manufacturing configuration 500B, pair of hooked prongs 32 is in contact with a portion of retention portion 28, and is ready to be slid proximally into securement with retention portion 28. For example, the sequence described with reference to FIGS. 5A to 5C may be reversed to cause pair of hooked prongs 32 to slide onto and about retention portion 28, and ultimately secure retention portion 28 between pair of hooked prongs 32. In particular, a distal force may be applied to wire 14 to cause pair of hooked prongs 32 to slide about and secure retention portion 28. When the distal force is applied, engagement portion 38 of implantable device 16 may engage against distal pusher end 22, and thus holding implantable device 16 in a fixed position relative to pusher 12 while wire 14 is moved relative to retention portion 28. For example, the distal force causes each prong of pair of hooked prongs 32 to bend or deform laterally outward of wire 14, increasing the inter-prong spacing from A1 to A2 (similar to that in FIGS. 5C and 5B) and allowing retention portion 28 to be received between pair of hooked prongs 32. When a maximum dimension of retention portion 28 passes proximally relative to pair of hooked prongs 32, each prong of pair of hooked prongs 32 bends or deforms laterally inward to reduce the increased inter-prong spacing A2 to the initial inter-prong spacing A1 (similar to that in FIGS. 5B and 5A).

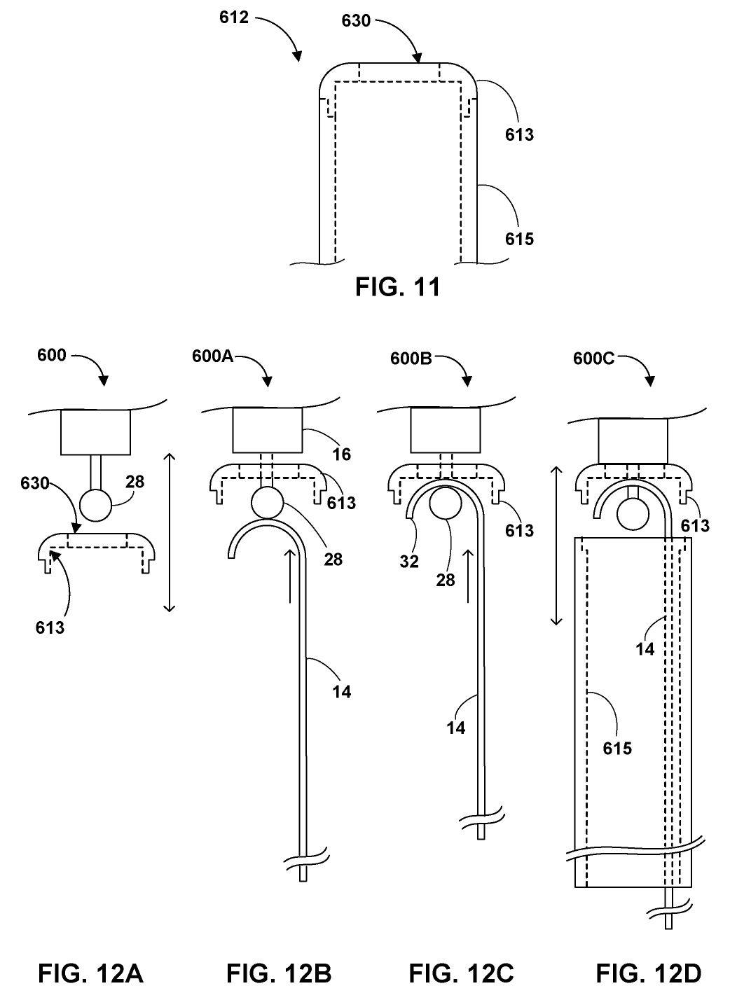

FIG. 10C is a conceptual diagram illustrating a partial side view of a final manufacturing configuration 500C of the implantation assembly of FIG. 11A. In final manufacturing configuration 500C, retention portion 28 is releasably secured between pair of hooked prongs 32. Implantation assembly 10 is now ready to be packaged and delivered to a clinician for use, for example, in a sterile package. The clinician may subsequently open the package, and deploy implantable device 16 using techniques described according to the present disclosure.

FIG. 11 is a conceptual diagram illustrating a partial side view of an example pusher 612 including a pusher cap 613 secured to a pusher body 615. Pusher cap 613 defines a pusher opening 630. Thus, after pusher cap 613 is secured to pusher body 615, pusher 612 is generally similar to pusher 12 described with reference to FIGS. 1A to 1G.

FIG. 12A is a conceptual diagram illustrating a partial side view of an initial manufacturing configuration 600 of an example implantation assembly 600 including pusher 612 of FIG. 11. Implantation assembly 600 may be similar to implantation assembly 10 described with reference to FIGS. 1A to 1G, except for including pusher 612 instead of pusher 12. In initial manufacturing configuration 600A, pusher cap 612 is ready to be moved relative to retention portion 28 such that retention portion 28 can be introduced across pusher opening 630.

FIG. 12B is a conceptual diagram illustrating a partial side view of a first intermediate manufacturing configuration 600A of implantation assembly 600A of FIG. 12A. In first intermediate manufacturing configuration 600A, pusher cap 613 is positioned between retention portion 28 and a remaining portion of implantable device 16. Wire 14 is ready to be secured to retention portion 28. For example, a distal force applied to wire 14 in configuration 600A causes each prong of pair of hooked prongs 32 to bend or deform laterally outward of wire 14, increasing the inter-prong spacing from A1 to A2 (similar to that in FIGS. 5C and 5B) and allowing retention portion 28 to be received between pair of hooked prongs 32. When a maximum dimension of retention portion 28 passes proximally relative to pair of hooked prongs 32, each prong of pair of hooked prongs 32 bends or deforms laterally inward to reduce the increased inter-prong spacing A2 to the initial inter-prong spacing A1 (similar to that in FIGS. 5B and 5A).

FIG. 12C is a conceptual diagram illustrating a partial side view of a second intermediate manufacturing configuration 600B of the implantation assembly of FIG. 12A. After the distal force is applied to the wire 14 in the first intermediate manufacturing configuration 600A, the second intermediate manufacturing configuration 600B is achieved in which wire 14 is secured to retention portion 28 (by applying the distal force to wire 14, similar to that described with reference to FIGS. 10A to 10C).

FIG. 12D is a conceptual diagram illustrating a partial side view of a third intermediate manufacturing configuration 600C of the implantation assembly of FIG. 12A. In In third intermediate manufacturing configuration 600C, pusher body 615 is ready to be secured to pusher cap 613 to form pusher 612. For example, pusher body 615 may be secured to pusher cap 613 by an adhesive, a weld, or overmolding (e.g., after softening a portion of pusher cap 613 and/or pusher body 615). After pusher body 615 is secured to pusher cap 613 to form pusher 612, the final manufacturing configuration of assembly 600 may resemble initial configuration of assembly 10 described with reference to FIGS. 1A to 1G. In the final manufacturing configuration, retention portion 28 is releasably secured between pair of hooked prongs 32 within a pusher lumen of pusher 612. Implantation assembly 600 is now ready to be packaged and delivered to a clinician for use, for example, in a sterile package. The clinician may subsequently open the package, and deploy implantable device 16 using techniques described according to the present disclosure.

While schemes for manufacturing assemblies are described with reference to assembly 10 in FIGS. 10A to 10C and assembly 600 in FIGS. 12A to 12D, any assembly according to the present disclosure may be assembled using similar schemes.

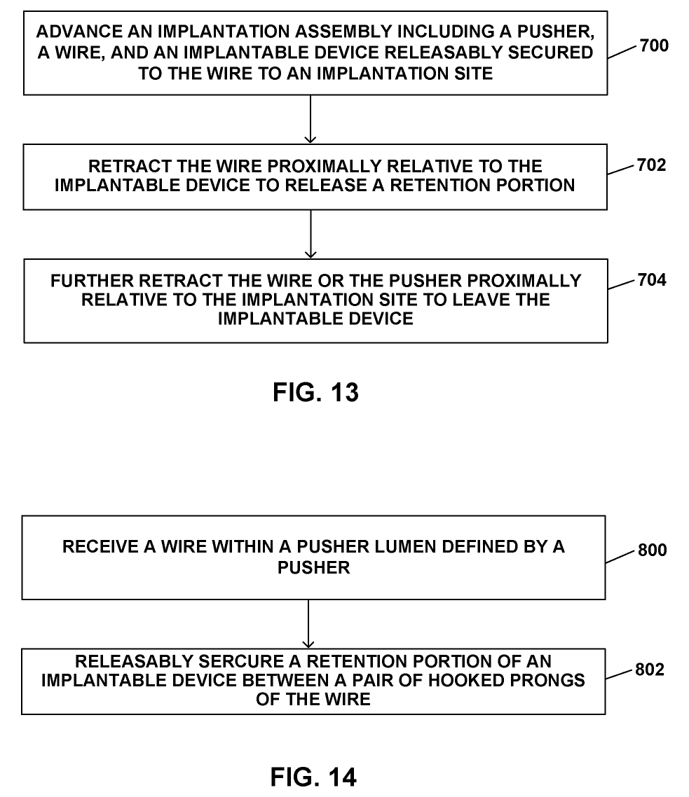

FIG. 13 is a flow diagram illustrating an example technique for delivering implantable device 16 to an implantation site using implantation assembly 10. While the example technique is described with reference to implantation assembly 10, any other implantation assembly according to the present disclosure may be delivered using the technique of FIG. 13.

In some examples, the technique includes advancing implantation assembly 10 to an implantation site (700). The technique may further include retracting wire 14 proximally relative to implantable device 16 (702) to release retention portion 28 from pair of hooked prongs 32 by causing pair of hooked prongs 32 to slide proximally about retention portion 28.

The technique may further include one or both of further retracting wire 14 or retracting pusher 12 proximally (704) relative to the implantation site to leave implantable device 16 released from pair of hooked prongs 32 at the implantation site.

FIG. 14 is a flow diagram illustrating an example technique for forming an implantation assembly. While the example technique is described with reference to implantation assembly 10 and implantation assembly 600, any other implantation assembly according to the present disclosure may be formed using the technique of FIG. 13. Further, implantation assemblies according to the present disclosure may be formed using any other suitable technique.

In some examples, the technique includes receiving distal portion 24 of wire 14 within pusher lumen 18 defined by pusher 14 (800). Pusher lumen 18 extends between proximal pusher end 20 and distal pusher end 22. Distal pusher end 22 defines distal pusher opening 30. Distal portion 24 of wire 14 defines pair of hooked prongs 32. The technique may further include receiving a retention portion of an implantable device through the distal pusher opening into the pusher lumen. The technique may further include releasably securing retention portion 28 of implantable device 16 between pair of hooked prongs 32 (802). The releasably securing retention portion 28 (802) may include exerting a force on pair of hooked prongs 32 in a distal direction to cause pair of hooked prongs 32 to distally slide about retention portion 28 of implantable device 16 and receive retention portion 28 between pair of hooked prongs 32. The releasably securing retention portion 28 (802) may include rotating pair of hooked prongs 32 relative to retention portion 28 to cause retention portion 28 to be received between pair of hooked prongs 32.

The technique may further include, before the releasably securing retention prong 28 (802), moving pusher cap 613 defining distal pusher opening 630 relative to retention portion 28 to cause retention portion 28 to pass proximally through distal pusher opening 630. The technique may further include, after the releasably securing retention prong 28 (802), securing pusher cap 613 to a distal portion of pusher 612 (e.g., to pusher body 615) to cause retention portion 28 and pair of hooked prongs 32 to be retained within a pusher lumen (e.g., of pusher 612).

The following enumerated clauses are described herein:

Clause 1: An implantation assembly configured to deploy an implantable device, the implantation assembly including: a pusher defining a pusher lumen extending between a proximal pusher end and a distal pusher end, the distal pusher end defining a pusher opening; and a wire extending within the pusher lumen, a distal portion of the wire defining a pair of hooked prongs, where the pusher opening is configured to receive a retention portion of the implantable device into the pusher lumen, and where the pair of hooked prongs is configured to releasably secure the retention portion of the implantable device within the pusher lumen.

Clause 2: The implantation assembly of clause 1, where at least a portion of the pair of hooked prongs is U-shaped.

Clause 3: The implantation assembly of any of clauses 1 or 2, where the pair of hooked prongs defines a pair of prong ends spaced apart from each other.

Clause 4: The implantation assembly of any of clauses 1 or 2, where the pair of hooked prongs defines a pair of prong ends secured to each other.

Clause 5: The implantation assembly of any of clauses 1 to 4, where at least a portion of the wire defines a flat wire cross-section or a round wire cross-section.

Clause 6: The implantation assembly of clause 5, where at least one hooked prong of the pair of hooked prongs defines a flat cross-section.

Clause 7: The implantation assembly of any of clauses 1 to 6, where the pair of hooked prongs is laterally movable between a first configuration in which the retention portion of the implantable device is secured between the pair of hooked prongs and a second configuration in which the retention portion of the implantable device is releasable from the pair of hooked prongs.

Clause 8: The implantation assembly of clause 7, where the pair of hooked prongs defines a first maximum inter-prong spacing in the first configuration, and where the pair of hooked prongs defines a second maximum inter-prong spacing greater than the first maximum inter-prong spacing in the second configuration.

Clause 9: The implantation assembly of clause 8, where the second maximum inter-prong spacing is at least 5% greater than the first maximum inter-prong spacing.

Clause 10: The implantation assembly of any of clauses 1 to 9, where each hooked prong of the pair of hooked prongs is identical in size and shape.

Clause 11: The implantation assembly of any of clauses 1 to 10, where a first hooked prong of the pair of hooked prongs extends along a first plane, and where a second hooked prong of the pair of hooked prongs extends along a second plane, where the second plane is parallel to the first plane.

Clause 12: The implantation assembly of any of clauses 1 to 10, where a first hooked prong of the pair of hooked prongs extends along a first plane, and where a second hooked prong of the pair of hooked prongs extends along a second plane, where the second plane is angled relative to the first plane.

Clause 13: The implantation assembly of any of clauses 1 to 12, where at least one hooked prong of the pair of hooked prongs extends along at least a partially curved path.

Clause 14: The implantation assembly of any of clauses 1 to 13, where at least one hooked prong of the pair of hooked prongs extends along at least a partially linear path.

Clause 15: The implantation assembly of any of clauses 1 to 14, where the wire extends along a wire path, and where at least one hooked prong of the pair of hooked prongs extends along a path parallel to at least a portion of the wire path.

Clause 16: The implantation assembly of any of clauses 1 to 15, where the wire extends along a wire path, and where at least one prong of the pair of hooked prongs extends along a path diverging laterally away from at least a portion of the wire path.

Clause 17: The implantation assembly of any of clauses 1 to 16, where the pusher opening is narrower than at least an engagement portion of the implantable device to resist entry of the engagement portion into the pusher lumen through the pusher opening.

Clause 18: The implantation assembly of any of clauses 1 to 17, where the pusher opening is narrower than the pair of hooked prongs to resist movement of the pair of hooked prongs distally beyond the pusher opening.

Clause 19: The implantation assembly of any of clauses 1 to 18, where the pusher further includes a pusher cap defining the distal pusher end and the pusher opening.

Clause 20: The implantation assembly of any of clauses 1 to 19, where the wire includes a metal or an alloy.

Clause 21: The implantation assembly of clause 20, where the alloy includes nitinol.

Clause 22: The implantation assembly of any of clauses 1 to 21, further including the implantable device.

Clause 23: The implantation assembly of any of clauses 1 to 22, where the pair of hooked prongs is configured to slidably receive a retention wire of the retention portion of the implantable device.

Clause 24: The implantation assembly of clause 23, where the retention portion includes a ball configured to be releasably secured by the pair of hooked prongs.

Clause 25: The implantation assembly of clause 24, where the pair of hooked prongs, in a first configuration in which the retention portion of the implantable device is secured between the pair of hooked prongs, defines a first maximum inter-prong spacing in a range of 30% to 70% of a maximum width of the ball.

Clause 26: The implantation assembly of clause 25, where the first maximum inter-prong spacing is in a range of 30% to 70% of a maximum lateral width of the pair of hooked prongs in the first configuration.

Clause 27: The implantation assembly of any of clauses 25 or 26, where the pair of hooked prongs, in a second configuration in which the retention portion of the implantable device is releasable from the pair of hooked prongs, defines a second maximum inter-prong spacing that is at least equal to the maximum width of the ball.

Clause 28: The implantation assembly of any of clauses 23 to 27, where at least one hooked prong of the pair of hooked prongs has a prong length at least twice a length of the retention wire.

Clause 29: The implantation assembly of any of clauses 22 to 28, where the implantation device further includes the engagement portion, where the engagement portion is distal to the retention portion, and where the engagement portion is wider than the pusher opening.

Clause 30: The implantation assembly of clause 29, where the engagement portion includes a radiopaque marker band.

Clause 31: The implantation assembly of any of clauses 22 to 30, where a composition of the retention portion differs from a composition of the pair of hooked prongs.

Clause 32: The implantation assembly of any of clauses 22 to 31, where the implantable device includes an implantable body including one or both of a spherical mesh or a helical coil.

Clause 33: The implantation assembly of clause 32, where the retention portion includes a steel, and where the wire includes nitinol.

Clause 34: A method including: advancing the implantation assembly of any of clauses 1 to 33 to an implantation site; and retracting the wire proximally relative to the implantable device to release the retention portion from the pair of hooked prongs by causing the pair of hooked prongs to slide proximally about the retention portion.

Clause 35: The method of clause 34, further including, after retracting the wire proximally: one or both of further retracting the wire or retracting the pusher proximally relative to the implantation site to leave the implantable device released from the pair of hooked prongs at the implantation site.

Clause 36: A method including forming the implantation assembly of any of clauses 1 to 33.

Clause 37: A method including: receiving a distal portion of a wire within a pusher lumen defined by a pusher, the pusher lumen extending between a proximal pusher end and a distal pusher end, the distal pusher end defining a distal pusher opening, the distal portion of the wire defining a pair of hooked prongs; receiving a retention portion of an implantable device through the distal pusher opening into the pusher lumen; and releasably securing the retention portion of the implantable device between the pair of hooked prongs.

Clause 38: The method of clause 37, where the releasably securing the retention portion includes exerting a force on the pair of hooked prongs in a distal direction to cause the pair of hooked prongs to distally slide about the retention portion of the implantable device and receive the retention portion between the pair of hooked prongs.

Clause 39: The method of any of clauses 37 or 38, where the releasably securing the retention portion includes rotating the pair of hooked prongs relative to the retention portion to cause the retention portion to be received between the pair of hooked prongs.

Clause 40: The method of any of clauses 37 to 39, further including: before the releasably securing the retention prong, moving a pusher cap defining the distal pusher opening relative to the retention portion to cause the retention portion to pass proximally through the distal pusher opening; and after the releasably securing the retention prong, securing the pusher cap to a distal portion of the pusher to cause the retention portion and the pair of hooked prongs to be retained within the pusher lumen.

Various examples have been described. These and other examples are within the scope of the following claims.

What is claimed is:

1. . An implantation assembly configured to deploy an implantable device, the implantation assembly comprising:
a pusher defining a pusher lumen extending between a proximal pusher end and a distal pusher end, the distal pusher end defining a pusher opening; and
a wire extending within the pusher lumen, a distal portion of the wire defining a pair of hooked prongs,
wherein the pusher opening is configured to receive a retention portion of the implantable device into the pusher lumen, and
wherein the pair of hooked prongs is configured to releasably secure the retention portion of the implantable device within the pusher lumen.

2. . The implantation assembly of claim 1, wherein at least a portion of the pair of hooked prongs is U-shaped.

3. . The implantation assembly of claim 1, wherein the pair of hooked prongs defines a pair of prong ends spaced apart from each other.

4. . The implantation assembly of claim 1, wherein at least one hooked prong of the pair of hooked prongs defines a flat cross-section.

5. . The implantation assembly of claim 1, wherein the pair of hooked prongs is laterally movable between a first configuration in which the retention portion of the implantable device is secured between the pair of hooked prongs and a second configuration in which the retention portion of the implantable device is releasable from the pair of hooked prongs.

6. . The implantation assembly of claim 1, wherein each hooked prong of the pair of hooked prongs is identical in size and shape.

7. . The implantation assembly of claim 1, wherein the wire extends along a wire path, and wherein at least one hooked prong of the pair of hooked prongs extends along a path parallel to at least a portion of the wire path.

8. . The implantation assembly of claim 1, wherein the pusher opening is narrower than at least an engagement portion of the implantable device to resist entry of the engagement portion into the pusher lumen through the pusher opening.

9. . The implantation assembly of claim 1, wherein the pusher opening is narrower than the pair of hooked prongs to resist movement of the pair of hooked prongs distally beyond the pusher opening.

10. . The implantation assembly of claim 1, wherein the pusher further comprises a pusher cap defining the distal pusher end and the pusher opening.

11. . The implantation assembly of claim 1, wherein the wire comprises a metal or an alloy.

12. . The implantation assembly of claim 11, wherein the alloy comprises nitinol.

13. . The implantation assembly claim 1, wherein the pair of hooked prongs is configured to slidably receive a retention wire of the retention portion of the implantable device.

14. . The implantation assembly of claim 13, wherein the retention portion comprises a ball configured to be releasably secured by the pair of hooked prongs.

15. . The implantation assembly of claim 14, wherein the pair of hooked prongs, in a first configuration in which the retention portion of the implantable device is secured between the pair of hooked prongs, defines a first maximum inter-prong spacing in a range of 30% to 70% of a maximum width of the ball.

16. . A method comprising:
advancing an implantation assembly to an implantation site, the implantable assembly comprising:
a pusher defining a pusher lumen extending between a proximal pusher end and a distal pusher end, the distal pusher end defining a pusher opening,
a wire extending within the pusher lumen, a distal portion of the wire defining a pair of hooked prongs, and
an implantable device comprising a retention portion releasably secured between the pair of hooked prongs; and
retracting the wire proximally relative to the implantable device to release the retention portion from the pair of hooked prongs by causing the pair of hooked prongs to slide proximally about the retention portion.

17. . The method of claim 16, further comprising, after retracting the wire proximally: one or both of further retracting the wire or retracting the pusher proximally relative to the implantation site to leave the implantable device released from the pair of hooked prongs at the implantation site.

18. . A method comprising:
receiving a distal portion of a wire within a pusher lumen defined by a pusher, the pusher lumen extending between a proximal pusher end and a distal pusher end, the distal pusher end defining a distal pusher opening, the distal portion of the wire defining a pair of hooked prongs;
receiving a retention portion of an implantable device through the distal pusher opening into the pusher lumen; and
releasably securing the retention portion of the implantable device between the pair of hooked prongs.

19. . The method of claim 18, wherein the releasably securing the retention portion comprises exerting a force on the pair of hooked prongs in a distal direction to cause the pair of hooked prongs to distally slide about the retention portion of the implantable device and receive the retention portion between the pair of hooked prongs.

20. . The method of claim 18, wherein the releasably securing the retention portion comprises rotating the pair of hooked prongs relative to the retention portion to cause the retention portion to be received between the pair of hooked prongs.
